(19) United States
(12) Patent Application Publication
(10) Pub. No.: US 2023/0352121 A1
(43) Pub. Date: Nov. 2, 2023

(54) COMPRESSED MULTI-SEQUENCE ALIGNMENT FOR POLYSACCHARIDE ARCHIVAL STORAGE

(71) Applicant: Dell Products L.P., Round Rock, TX (US)

(72) Inventors: Ofir Ezrielev, Be'er Sheba (IL); Jehuda Shemer, Kfar Saba (IL)

(21) Appl. No.: 17/660,904

(22) Filed: Apr. 27, 2022

(51) Int. Cl. G16B 50/50; G06F 16/11

(52) U.S. Cl. CPC G16B 50/50; G06F 16/113

(57) ABSTRACT

One example method includes encoding data as a polysaccharide structure, synthesizing the polysaccharide structure to create polysaccharide storage media that comprises the data, and storing the polysaccharide storage media. The example method may also include compressing the polysaccharide and storing the compressed data as a polysaccharide.

### FIELD OF THE INVENTION

Embodiments of the present invention generally relate to archive storage. More particularly, at least some embodiments of the invention relate to systems, hardware, software, computer-readable media, and methods for the use of polysaccharides for archival data storage and associated IO operations.

### BACKGROUND

Currently, archival data storage typically employs magnetic tapes or disks drives. Due to recognized problems with these approaches, attention has turned to the chemical storage. An example of chemical storage is to DNA (deoxyribonucleic acid) storage. While DNA storage technology is advancing, it has a number of disadvantages.

For example, DNA has 4 states, which is only double those of a computer bit which can assume values of either ‘0’ or ‘1.’ As another example, DNA requires special storage conditions to maintain its stability. One approach is the encapsulation of DNA within an inorganic matrix comprised of silica, iron oxide, or a combination of both. Some estimate that encapsulation in silica particles could maintain DNA for 20-90 years at room temperature, 2000 years at 9.4° C., to over 2 million years at −18° C. However, there are several potential limitations to consider.

First, the physical processes of encapsulation and retrieval take time. Second, the encapsulation of the DNA inherently reduces the information density of the storage system. A layer by layer design with alternating DNA and cationic polyethylenimine with a silica final encapsulation has achieved the best storage density to date in such systems, ˜3.4 weight % DNA. However, this is a sacrifice of 1-2 orders of magnitude in information density, which is a significant limitation.

Chemical storage is a nascent storage technology that may provide benefits in areas including data compression.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings, in which:

FIG. 1A discloses aspects of an example monosaccharide that may be employed in example embodiments;

FIG. 1B discloses some example enantiomers that may be employed in example embodiments;

FIG. 2 discloses aspects of a glucose-6-phosphate molecule that may be employed in some embodiments of the invention;

FIG. 3 discloses a table comparing the properties of various biopolymers;

FIG. 4 discloses examples of a chain polysaccharide, and a branched polysaccharide, such as may be employed in some embodiments of the invention;

FIG. 5 discloses an example of a branched polysaccharide attached to a protein, that may be employed in some example embodiments of the invention;

FIG. 6 is an example method according to some embodiments of the invention;

FIG. 7 discloses aspects of a compression engine configured to compress data;

FIG. 8 discloses aspects of a compression engine configured to compress data using multiple sequence alignment;

FIG. 9 discloses aspects of compressing data that include long zero sequences;

FIG. 10 discloses aspects of pointer pairs;

FIG. 11 discloses aspects of warming up operations to reduce computation times in compression operations;

FIG. 12 discloses aspects of hierarchical compression;

FIG. 13 discloses aspects of performing compression in a computing environment;

FIG. 14 discloses aspects a glycosidic bond and representing or encoding data in a polysaccharide;

FIG. 15 discloses aspects of compressing data for polysaccharide storage;

FIG. 16 discloses aspects of compressing a polysaccharide;

FIG. 17 discloses aspects of compressing a polysaccharide that includes branches; and

FIG. 18 discloses a computing device or system or entity operable to perform and/or control the performance of, any of the disclosed methods, processes, and operations.

### DETAILED DESCRIPTION OF SOME EXAMPLE EMBODIMENTS

Embodiments of the present invention generally relate to archive storage. More particularly, at least some embodiments of the invention relate to systems, hardware, software, computer-readable media, and methods for the use of polysaccharides for archival data storage and associated IO operations.

Embodiments of the present invention further relate to compression and compression operations using data alignment. More particularly, at least some embodiments of the invention relate to systems, hardware, software, computer-readable media, and methods for compressing data and further relate to compressing data using sequence alignment.

Embodiments of the invention provide a compression engine that is configured to compress data using an alignment mechanism. The compression engine receives a file or data as input and performs a splitting operation to generate a matrix of sequences. The file is split into multiple sequences. Each sequence corresponds to part of the file being compressed. When the matrix is generated, gaps may be included or inserted into some of the sequences for alignment purposes. Once the matrix is completed, a consensus sequence is identified or derived from the compression matrix. The original file is compressed by representing the input file as a list of pointer pairs or pointer lists into the consensus sequence. Each pointer pair or list corresponds to a part of the data and each pointer pair or list identifies the beginning and end of a subsequence in the consensus sequence. The file can be reconstructed by concatenating the subsequences in the consensus sequence identified by the pointer pairs or lists.

Embodiments of the invention are discussed with reference to a polysaccharide or other biological sequences or structures, which may include nucleic acids by way of example and not limitation. The compression operations discussed herein may be applied to any data or data type. Embodiments of the invention, in addition to polysaccharide storage, further relate to MSA (Multi-Sequence Alignment) based compression in the context of polysaccharide storage.

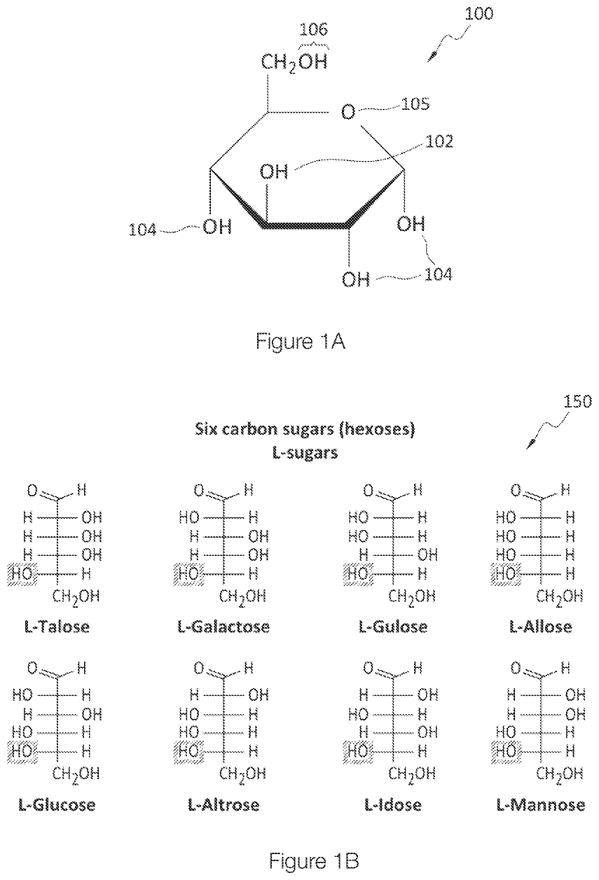

FIG. 1A-7 disclose aspects of polysaccharide storage. In one example embodiment, a polysaccharide, which may be in a chain or branched form, is synthesized whose particular structure embodies an encoding of data. The synthesis process thus constitutes a write operation. The encoded data may later be read out, such as in response to an IO, by mapping out the structure of the polysaccharide and then traversing the mapped structure.

A polysaccharide that encodes data may be relatively stable and robust over a range of environmental conditions. An embodiment may implement data storage in a polysaccharide whose storage capacity is one, two, or more, orders of magnitude larger than binary or DNA storage.

As noted in https://en.wikipedia.org/wiki/Polysaccharide, “Polysaccharides are the most abundant carbohydrate found in food, and some are already used widely in the industry for many uses (other than nutrition). Examples include [energy] storage polysaccharides such as starch, glycogen and galactogen and structural polysaccharides such as cellulose and chitin. They are long chain polymeric carbohydrates composed of monosaccharide units bound together by glycosidic linkages. This carbohydrate can react with water (hydrolysis) using amylase enzymes as catalyst, which produces constituent sugars (monosaccharides, or oligosaccharides). They range in structure from linear to highly branched. Polysaccharides are often quite heterogeneous, containing slight modifications of the repeating unit. Depending on the structure, these macromolecules can have distinct properties from their monosaccharide building blocks.”

DNA digital data storage is the process of encoding and decoding binary data to and from synthesized strands of DNA. While DNA as a storage medium may have significant potential because of its high storage density, its practical use is currently severely limited because of its high cost and very slow read and write times, although as of 2019, write times had improved to about 4 Mb/s.

While DNA has shown some promise as a storage medium, the polysaccharide data storage embraced by example embodiments of the invention is simpler, denser and has the potential to surpass DNA as a storage medium.

The data era is characterized by an overwhelming amount of data that is being generated and stored. As the amount of data collected, managed, and analyzed in a modern data center keeps growing at an exponential rate, the need for new and better storage methods is generally acknowledged.

Of the data collected, regulatory and various other reasons necessitate vast amounts of archival storage. Currently such archival storage is done with disks, which experience shortages and, in addition, their manufacture requires the mining of rare earth metals, and the industrial processes involved in such mining may severely harm the environment. Another solution which is being developed is DNA storage, which will become a better solution in the long run but is not without its faults.

In light of considerations such as these, example embodiments are directed to a form of next-generation data storage using polysaccharides, sometimes referred to as ‘long sugars’ as they may take the form of chain structures or branch structures that include multiple monosaccharides connected together. When employed as a data storage medium, polysaccharides may provide greater storage and better stability than DNA storage. Moreover, polysaccharides are chemically and physically stable and do not require special storage conditions. For example, polysaccharides may be reliably stored in the same types of environments, for example, with regard to moisture and temperature ranges, that are recommended for magnetic or silicon-based storage. Further, polysaccharide archival data storage according to example embodiments may take the form of thin layers that can be efficiently stored.

In general, example embodiments are directed to polysaccharide data storage media at solid state for data archiving. Polysaccharides may be particularly well suited for data storage due to the complexity possibilities of polysaccharides and the ease of their maintenance in solid form. Example embodiments may provide various functionalities in connection with polysaccharide data storage media. These functionalities include: (1) write operations, and addressing, for the polysaccharide data storage media; (2) storage/maintenance of the polysaccharide data storage media storage media; and (3) read operations, that is, reading data from the polysaccharide data storage media. Note that while the description herein covers basic operations of storage, all existing RAID technologies may be applied directly to this solution.

With reference now to FIG. 1A, one example of a monosaccharide sugar that may be used for example embodiments is glucose, whose chemical structure is denoted at 100. With the CH₂OH direction as reference, note that the OH groups can either face in its direction or opposite to it. As there are 4 such OH groups, glucose has 2⁴, that is, 16, enantiomers, each with distinct respective structural, optical, and biological characteristics. Put another way, each of the OH groups may be analogized to a bit that has one of two positions, namely, each OH group extends either (1) in the direction of the CH₂OH, as in the case of OH group 102, or (2) away from the CH₂OH, as in the case of OH groups 104.

Any number of monosaccharides, such as the glucose 100 for example, can then be linked to each other via glycosidic bonds to create more complex compounds referred to as polysaccharides, known examples of which are glycogen, cellulose and starch. With reference again to the example of FIG. 1, there are 5 OH groups that can participate in the glycosidic bond from each monosaccharide to another (1-4 and 6, as the 5th oxygen 105 is a part of the main ring), that is, the OH groups 102, 104, and 106.

Thus, for a single polysaccharide chain of glucose enantiomers only, its representation power can be compared to that of a bit sequence and DNA sequence.

Table 1, below, illustrates the possibilities of chemical storage compared to conventional storage

TABLE 1

| | Representation power-n-length |
|---|---|
| Method | sequence |
| Bit sequence | 2ⁿ |
| DNA sequence | 4ⁿ |
| Polysaccharide sequence | 16ⁿ(enantiomers) * 5ⁿ * 4ⁿ⁻²(bonds) |

As shown in Table 1, a bit sequence with ‘n’ positions can represent 2ⁿ possibilities, and a DNA sequence with 4 possible values for each of ‘n’ positions can represent 4ⁿ possibilities. In contrast, the representation power of a polysaccharide sequence, that is, the amount of data that can be represented by a polysaccharide sequence, with ‘n’ monosaccharides is significantly greater than that of a bit sequence or a DNA sequence. It was noted earlier that glucose has 16 enantiomers, due to the fact that it has 4 OH groups, each of which can assume 2 different orientations (2⁴). Given that, the 4 OH groups can collectively define 16 different configurations, or enantiomers, of the glucose 100. Thus, with reference again to Table 1, the total number of representations possible with a group of ‘n’ monosaccharides is the number of enantiomers (16ⁿ)×the number of possible bonds (5ⁿ4ⁿ⁻²). Note that there are typically 4 options on the initiator, as one of the 5 is taken by the last bond, unless it is the first monosaccharide, or the last one which is not an initiator, and 5 on the second monosaccharide in the bond.

This approach results in a base 320 numeral system, in which each digit in a numeral can have any of 320 different values. In contrast, a conventional bit sequence is a base 2 system where each bit can be 0 or 1, and DNA is a base 4 system. Thus, an ability of some example embodiments to represent data is at least 2 orders of magnitude greater than the respective abilities of a bit, or DNA, to represent data. Following is a discussion of some example 10 operations that may be performed by various embodiments of the invention.

FIG. 1B discloses some other example enantiomers 150 that may be employed in some embodiments of the invention. It is noted that no particular enantiomer(s) are required to be used in any embodiment.

First, it may be determined how to represent information held in the polysaccharide sequence. For the sake of simplicity, and continuing with the example noted above, some example embodiments may employ a representation power 16ⁿ⁻¹ (enantiomers)*5ⁿ⁻¹*4ⁿ⁻¹ (bonds). Thus, embodiments may use 16*5*4=320-base numeral system. At this point, it is a matter of moving from a number from a binary base to a 320-base. Consequently, each numeral in the resulting number represents a specific monosaccharide enantiomer and the bond details to the next monosaccharide. Thus, the polysaccharide sequence embodying the data to be written is obtained.

After the needed polysaccharide sequence has been determined, that sequence must then be synthesized. Details concerning the synthesis of some example polysaccharides can be found at: https://pubs.acs.org/doVabs/10.1021/jacs.0c00751, which is incorporated herein in its entirety by this reference. Briefly summarized, a synthesizer method and system may be used to perform an AGA (Automated Glycan Assembly) process to synthesize polysaccharides such as may be employed in some example embodiments of the invention. Currently, polysaccharide synthesis using the AGA approach may take hours, but the speed is rapidly improving and, with parallelism, sufficient speeds for data archiving applications are expected by some to be only a few years in the future.

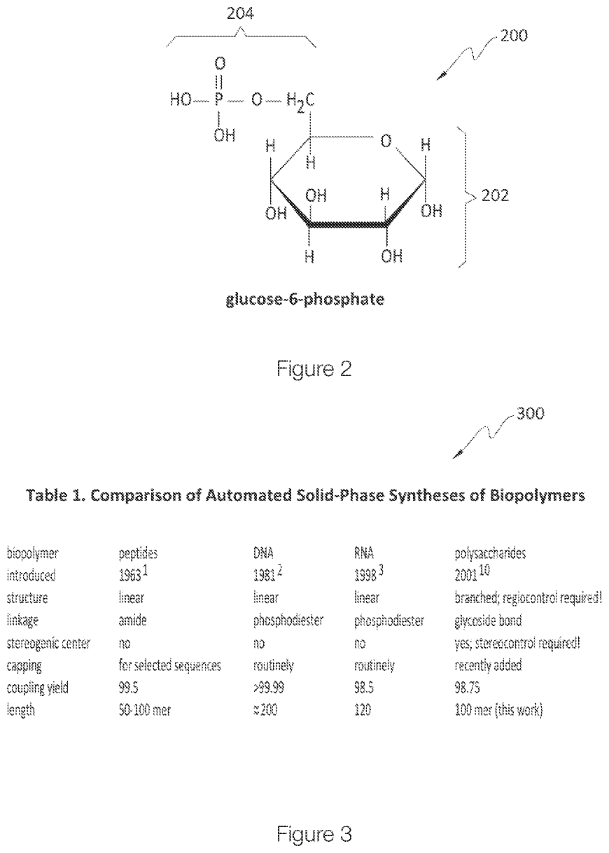

With reference now to FIG. 2, a glucose-6-phosphate molecule 200 is shown. In this illustrative embodiment, the first monosaccharide 202 is labeled for use as a point of reference for later reading as the starting point. That is, data represented by a polysaccharide that includes the monosaccharide of FIG. 2 may be read out by traversing the polysaccharide beginning at the labeled starting point. In the example of FIG. 2, the first monosaccharide 202 is labeled with a phosphate group 204 connected to the 6th carbon. However, different labels and/or locations of labels may be employed in other embodiments. Thus, the example of FIG. 2 is provided for the purposes of illustration and is not intended to limit the scope of the invention in any way.

In the example of FIG. 2, an ‘n’ length bit sequence can be represented by a polysaccharide sequence of length L thus:

L=[n/(log₂(320))+1], where

log₂(320) is approximately 8.322.

By comparison, an ‘n’ length bit sequence would require a DNA sequence length of L=[n/2] for representation.

With reference now to FIG. 3, a table 300 is disclosed that compares properties of various biopolymers, including the capped sizes for chemical synthesis, before merging into large units such as chains or branched configuration, of the different biopolymers. As shown in the table 300, the amount of information in a 100-mer polysaccharide sequence, that is based on the example monosaccharide disclosed herein that employs only 16 enantiomers, is equivalent to a DNA sequence ˜415 long. Thus, the 100-mer polysaccharide sequence presents 2.1×(415/200) improvement over the current 200 length of the DNA sequence.

Embodiments of the invention include various configurations of a polysaccharide data storage entity. Such configurations include, for example, a chain of monosaccharides connected together to define a polysaccharide data storage entity. Another example configuration of a polysaccharide data storage entity comprises a branched arrangement of monosaccharides. Further, some embodiments of a polysaccharide storage entity may be ‘flat,’ that is, two dimensional, while other embodiments of a polysaccharide storage entity may be three dimensional. It is noted that the scope of the invention is not limited to any particular monosaccharide, or polysaccharide, form or configuration.

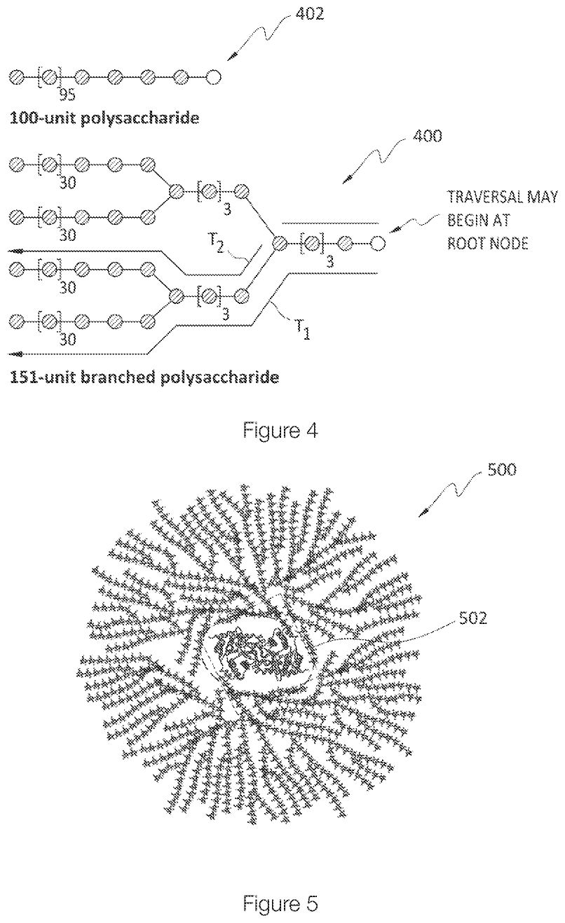

Polysaccharide synthesis techniques, such as AGA for example, may enable creation of highly branched polysaccharides. Illustrative examples of a branched polysaccharide 400, and chain polysaccharide 402, are disclosed in FIG. 4. As shown in FIG. 4, the tree structure of the polysaccharide 400 imposes a particular order on the molecules that make up the polysaccharide 400. This particular order, which may be specified as part of the polysaccharide 400 synthesis process, may embody particular data when the tree structure is traversed as part of a data read process.

One or more traversals of the tree structure, such as implemented by the polysaccharide 400, may be employed as part of a data read process. For example, a traversal may begin at the root of the tree, and then follow all branches to the left, before returning to the root or another starting point and next traversing, for example, the branches to the right. The order in which the tree is traversed may thus define particular data. Accordingly, a single tree may represent various different data, depending upon the particular order(s) in which that tree is traversed. For example, a particular traversal of a tree may define a particular file, or object. It is noted that the scope of the invention is not limited to any particular tree, tree size or structure, traversal order, or traversal process.

FIG. 5 discloses an alternative embodiment of a branched polysaccharide 500, specifically, glycogen, connected to a protein 502. The protein 502 may serve as a starting point for addressing. More specifically, this example branched polysaccharide 500 can be ‘flattened’ by a BFS (breadth first search) traversal or DFS (depth first search) traversal of the labeled monosaccharide. In a BFS process, also sometimes referred to as a level order traversal, the tree or other structure is traversed, starting at a root node for example, level by level so that all nodes of a level are traversed before the process moves to the next lower level, where the process is repeated. In a DFS process, the structure is traversed, starting from the root node for example, and the nodes explored as far as possible, such as by traversing, as shown by T₁ in FIG. 4, to the left of the root node and continuing to traverse left at each node, until a node is reached that does not have any unvisited adjacent nodes. At this point, the traversal process may backtrack to the root node, or other traversal starting point, and then traverse as shown by T₂ in FIG. 4, and the traversal process may continue until all the nodes have been visited.

Branched polysaccharides may enable the use of relatively stabler and more compact polysaccharide structures. Moreover, branched polysaccharides provide an addressing system to data location, that is, the DFS traversal sequence is deterministic and linear and can thus provide a “tape” like addressing system. In effect, the branches in the polysaccharide structure serve as elements of the addressing system, since each branch guides the traversal process in a particular direction to a particular destination.

As polysaccharides are typically very stable in solid form, there may not be any special requirements to their storage. Polysaccharides may, for example, be saved as layers or a ‘sugar ball’ or ‘sugar cube’ weighing only a few grams. When archiving very large amounts of data, it may be necessary to have more than one ‘sugar ball’ or ‘sugar cube’ to ensure that all the data is represented. In such cases, the sugar balls may be placed in ordered compartments, or stored in any other suitable manner, to signify their order relative to each other. In this way, the data encoded in the polysaccharides may be read out in the correct order. Polysaccharides are generally stable in temperatures well below freezing and above ˜70° C., although the threshold temperatures may vary from one polysaccharide to another. Moisture conditions that can be tolerated by polysaccharides may be similar to those defined for the safe storage of conventional magnetic and electronic media. Moreover, polysaccharide data storage are resistant to magnetic fields and other phenomena that may damage conventional magnetic and electronic media and may corrupt the data stored on such conventional media. Finally, polysaccharide data storage may be resistant to unauthorized access since it cannot be read or accessed with the devices typically used to read conventional magnetic and electronic storage media.

In general, a data read operation may be performed by mapping out the structure, or topology, of the polysaccharide. After the structure is mapped, the starting point, such as the root node in FIG. 4 for example, is detected, and a traversal can be performed beginning at that starting point.

In more detail, and given a solid form, such as a ‘sugar ball’ for example, of the polysaccharide, an example read process may comprise, first, determining the structure of the polysaccharide. This determination of the structure may be performed using, for example, an NMR (nuclear magnetic resonance) imaging process, or a crystallography process such as may be performed with an X-ray device, or any other device capable of determining the structure of a polysaccharide. The structure may then be traversed and, as a result of the traversal, an X-base number may be determined that corresponds to the path traversed, where X may be 320, in some example embodiments. Note that the scope of the invention is not limited to any particular base system, and reference to a 320-base system is only by way of example, and not limitation.

Next, the resulting X-base number may be converted to its binary representation, which is the data original binary sequence, that is, the data, that was encoded in the polysaccharide. It should be noted that depending on the reading technique employed, it is possible that some amount of the storage material, that is, the polysaccharide, may be lost in the process. This problem may be addressed by making larger ‘sugar balls’ or chunks than are needed to store the data. These larger chunks would allow for multiple read cycles, and the corresponding losses of storage material that could occur with each read cycle. Because some embodiments are directed to polysaccharide archival data storage, it may be the case that the stored data will be read only rarely, if ever.

As noted earlier, a polysaccharide data storage medium may take the form of ‘sugar cubes’ or ‘sugar balls,’ for example. It that sense they are used similarly to the way a tape cassette or CD are used, that is, there is a device to write the data. The result is a cube or other form of data storage that a user can eject from the write device and store elsewhere. To read the polysaccharide data storage media, the user may “load” the cube or other form of polysaccharide data storage media into a read device and read out the data. Note that this is different from how a magnetic disk or SSD, for example, are used. This also means that when reading, the technology used to read the storage media may have advanced during the time when that media was in storage. This is particularly likely where archive media is concerned since a relatively long period of time may pass between the time when the archive media is stored, and the time when it is subsequently accessed. Thus, there is no direct connection between the write and read processes. In the same way, a new and faster CD device can still read media produced by older and slower write devices. Because archival storage is often targeted for years, depending on current read technology for later retrieval is problematic. DNA storage also shares the advantage that it will be readable in the future even after read technologies have advanced. On the other hand, media such as VHS cassettes have the problem that they are not readable by current read technologies, and it is a major industry issue converting all data to updated mediums every few years.

As will be apparent from this disclosure, example embodiments of the invention, which include polysaccharide data storage media, may provide various features and benefits. For example, embodiments of the invention include a system to represent data as a polysaccharide sequence for data archiving. As another example, when compared to drives (bits) or DNA (base pairs), polysaccharide data storage media provide a much larger representation power, and thus can store more data in the same sequence length. Further, when compared to DNA storage, polysaccharide storage media according to example embodiments are more stable and thus require significantly less storage maintenance. In addition, polysaccharide molecules are smaller than nucleic acid molecules. As a final example, embodiments of the invention may enable restoration of vast amounts of data without the use of any metals or other possibly hazardous or rare materials. As such, example embodiments may provide archival data storage media that implies only a minimal environmental footprint.

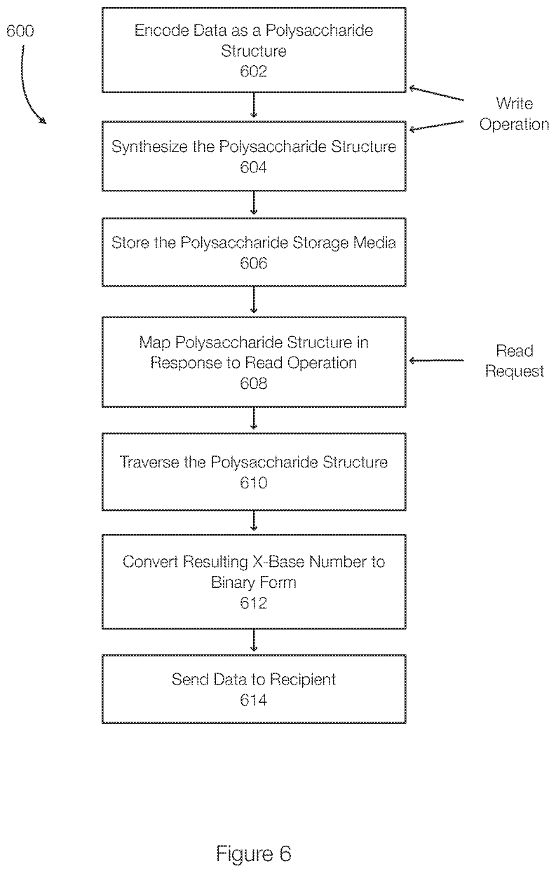

It is noted with respect to the example method of FIG. 6 that any of the disclosed processes, operations, methods, and/or any portion of any of these, may be performed in response to, as a result of, and/or, based upon, the performance of any preceding process(es), methods, and/or, operations. Correspondingly, performance of one or more processes, for example, may be a predicate or trigger to subsequent performance of one or more additional processes, operations, and/or methods. Thus, for example, the various processes that may make up a method may be linked together or otherwise associated with each other by way of relations such as the examples just noted. Finally, and while it is not required, the individual processes that make up the various example methods disclosed herein are, in some embodiments, performed in the specific sequence recited in those examples. In other embodiments, the individual processes that make up a disclosed method may be performed in a sequence other than the specific sequence recited.

Directing attention now to FIG. 6, an example method 600 is disclosed. Portions of the method 600 may be performed using processes and equipment such as, but not limited to, AGA processes and associated equipment, X-ray processes and associated equipment, and crystallography processes and associated equipment. The operation of these various types of equipment may be controlled by a processor executing instructions that are carried on a non-transitory computer readable storage media.

The method 600 may comprise a data write operation, or simply a ‘write operation,’ which may include encoding data as a polysaccharide structure 602. That is, the specific polysaccharide structure uniquely embodies the data. After the data has been encoded 602 as a particular polysaccharide structure, the polysaccharide structure that embodies the data may then be synthesized 604. Thus, in some embodiments, the operations 602 and 604 together comprise a write operation. The polysaccharide storage media that was created at 604 may then be stored 606. In some embodiments, the polysaccharide storage media that was created at 604 may comprise archive data storage, although the scope of the invention is not limited to the use of polysaccharide storage media as archive data storage.

At some later point in time after the polysaccharide storage media has been stored 606, a read request may be directed to, and received by, a controller or other element in communication with the polysaccharide storage media. In response to receipt of the read request, the polysaccharide structure may be mapped 608. This mapping 608 may comprise, for example, generation of a graphical and/or other representation of the physical polysaccharide structure. After the polysaccharide structure has been mapped 608, the map of the polysaccharide structure may be traversed 610 to obtain a particular number, such as an X-base number for example. The X-base number may then be converted 612 to its binary representation, which is the original binary sequence, that is, the data, that was encoded in the polysaccharide structure. Finally, the data may then be sent to 614 to the requestor.

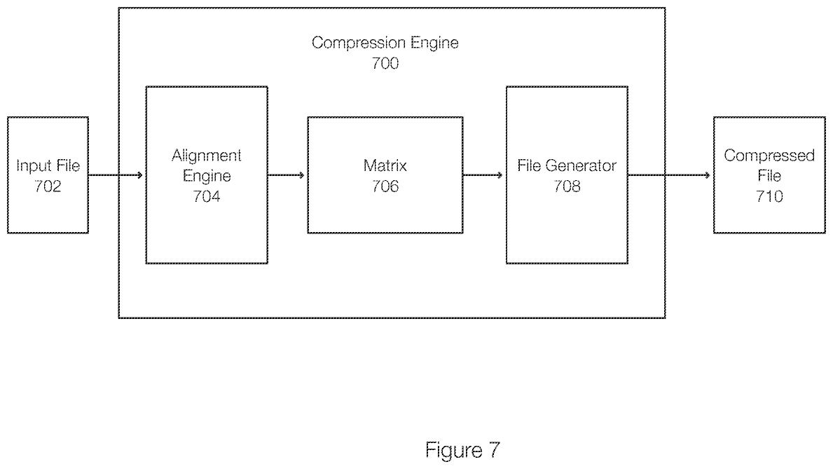

FIGS. 7-13 illustrate aspects of compression using multiple sequence alignment. When compressing data based on sequence alignment, one object of alignment is to divide the data into multiple pieces that are similar. The goal of alignment is to identify sequences that are similar. For example, the similarity of sequences can be scored. In a situation where n bits of a file are represented by a letter, a sequence of ABABAB may be similar to a sequence of ABAAAC. A similarity score may reflect, for example, how many of the letters match. In one example, similar sequences can be made to be identical by inserting gaps into the sequences. Identical, in this context, means that the columns of the sequences, when arranged in a matrix, have the same letters (or a gap).

More specifically, in order to achieve the alignment, it may be necessary to adjust some of the sequences such that each piece of the file includes data represented in a manner that allows the pieces or sequences to be aligned. For example, assume a file is represented by AAAAAAB. Also assume that the file is split into two pieces (or sequences): AAA and AAB. In order to align these sequences, it is necessary to insert a space or a gap. This may result in the following sequences (a space or gap is represented by “-”): AAA- and AA-B. When these sequences are aligned in a matrix, each column contains the same letter and/or gaps. This allows the file to be compressed as more fully described below.

The alignment process results in multiple sequences that can be arranged in a matrix in one example. Because a file may be large, the alignment process may involve an iterative process of splitting and aligning. This is performed until the length of the sequences is sufficient or when a consensus length has been reached.

By way of example, the alignment process maintains a list of pieces that can be split. These pieces may have the same length in one example. For each round or iteration, a piece is selected. In an embodiment, a piece with the highest length is split, and aligned. If the consensus length is smaller by a determined threshold than the previous consensus length, new pieces or sequences resulting from the split are added back to the list of pieces that can be split. This process continues until resources are exhausted, the length of the consensus is sufficient (e.g., meets a threshold length), or the list of splitable pieces is empty. This process may also include adding spaces or gaps as appropriate. In one example, gaps are added in each alignment. When completed, a compression matrix is generated as discussed below.

FIG. 7 discloses aspects of compressing data with a compression engine. FIG. 7 illustrates a compression engine 700. The compression engine 700 may be implemented at the edge, at the near edge, or in the cloud and may include physical machines (e.g., servers), virtual machines, containers, processors, memory, other computing hardware, or the like or combination thereof. The compression engine 700 may cooperate with or be integrated with a system or application such as a data protection system. For example, data backups, volumes, disk/volume images, or the like may be compressed prior to transmission over a network, prior to storage, for archiving, or the like. In some examples, compression operations are examples of data protection operations.

The compression engine 700 is configured to receive a file 702 as input. The compression engine 700 outputs a compressed file 710. More specifically, the file 702 is received at an alignment engine 704 that is configured to generate a compression matrix 706. In one example, the alignment engine 704 may perform a greedy splitting algorithm on the file 702 to generate the matrix. The splitting algorithm, in effect, divides the bits of the file 702 into multiple sequences of the same length. After each split, the alignment of the pieces is evaluated. If not sufficiently aligned, one or more of the pieces may be split again. This process may continue until the remaining pieces of sequences are sufficiently aligned. Once aligned, the resulting sequences constitute the compression matrix 706 and each sequence may correspond to a row of the matrix 706. If necessary, gaps are inserted into some of the sequences such that the matrix 706 is aligned. Gaps may be inserted during the alignment process.

More specifically, the matrix 706 may be represented a structure that includes rows and columns. The alignment engine 704 may be configured to determine the number of columns and/or rows during the splitting or alignment operation. During alignment, the file 702 is split until the rows of the matrix 706 can be generated. The alignment performed by the alignment engine 704 ensures that, for a given column in the matrix 706, the entries are all the same, except that some of the entries in a given column may be gaps. As previously stated, during alignment, gaps may be inserted at various locations of the sequences such that each column contains the same information in each row or a gap.

A consensus sequence is identified from the matrix 706 or derived from the matrix 706 and used by the file generator 708 to generate the compressed file 710. The entire file 702 is represented in the consensus sequence. Because each of the rows correspond to a part of the file and each has information that is present in the consensus matrix, the bits in the file can be represented using pointers into the consensus sequence. The compressed file 710 may include the consensus sequence and the pointer pairs. Each row of the compression matrix may be represented by one or more pointers. Gaps in a given row are not represented by pointers. Once the compressed file 710 is generated, the compression matrix 706 may be discarded.

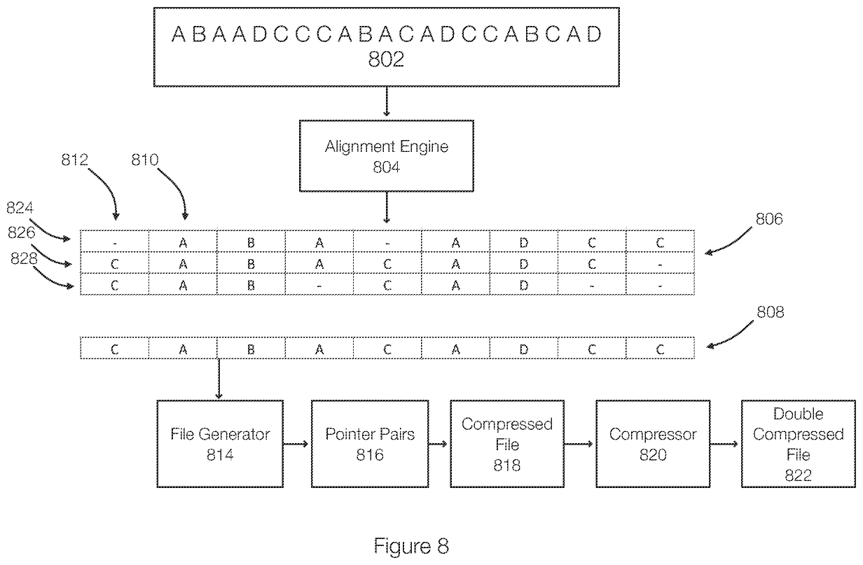

FIG. 8 discloses aspects of compressing a file. In FIG. 8, a file 802 is illustrated or represented as a series of letters: ABAADCCCABACADCCABCAD. Each of these letters may represent n bits of the file 802. Because n may vary or change from one compression operation to the next compression operation, the compression ratio may also change. In one example, n may be specified as an input parameter to the alignment engine 804 or may be determined by the sequencing or aligning performed by the alignment engine 804. The size of n may impact computation time.

The file 802 is aligned (or sequenced) by the alignment engine 804 to generate a compression matrix 806. The compression matrix includes rows and columns. Each column, such as the columns 810 and 810, contain either the same letter and/or a gap, which gap is represented as a “-” in FIG. 8. During sequencing or alignment performed by the alignment engine 804, the file 802 may be split into pieces until the matrix 806 is generated. When the alignment engine 804 completes its work and the pieces of the input file 802 are aligned, each of the columns in the matrix 806 contains the same letter and/or a gap. Thus, each row of the matrix 806 of the column 812 include the letter “C” and a gap while the column 810 contains the letter “A” with no gaps. No mismatches (e.g., a column contains more than one letter) are allowed.

The alignment performed by the alignment engine 804 allows a consensus sequence 808 to be generated or determined. The consensus sequence 808 includes the letters of the corresponding columns from the matrix 806. In this example, the consensus sequence 808 is generated from the matrix 806. However, the matrix 806 may also include the consensus sequence 808.

In effect, the consensus sequence 808 is a vector v, where v[i] is the letter or letter type that exists in column i, disregarding gaps. The vector may be multi-dimensional when compressing multi-dimensional data.

The pseudocode performed by the alignment engine 804 is as follows:

```
input: file V, with each k bits represented as a single letter
set splitCandidates ←{V}
set nonSplit ←{ }
while |splitCandidates| > 0:
  baseCMSA ← CMSA(nonSplit ∪ splitCandidates)
  set splitCandidates_{new} ←{ }
  set nonSplit_{new} ← nonSplit
  for volumePiece in splitCandidates: //Can be done concurrently
    L, R ← halve volumePiece
    if len(CMSA(nonSplit ∪ splitCandidates\volumePiece ∪ L ∪ R)) <
len(baseCMSA):
      splitCandidates_{new} = splitCandidates_{new} ∪ L ∪ R
    else:
      nonSplit_{new}= nonSplit_{new} ∪ volumePiece
  splitCandidates = splitCandidates_{new}
  nonSplit = nonSplit_{new}
```

Once completed, the nonSplit sequences will be a matrix of letters and gaps, such as the matrix 806. The consensus sequence 808 is taken or derived from the matrix 806.

The file generator 814 uses the consensus matrix 808 to generate pointer pairs that represent the letter or bits in the file. In this example, the consensus matrix 808 is an array or vector with entries 0 . . . 8. When generating the pointer pairs, the matrix 806 may be processed row by row. In the first row, the first subsequence is ABA corresponds to locations 1, 2, and 3 of the consensus sequence 808. The first pointer in the list of pointer pairs 816 is thus P₁ (1:3).

Using the consensus matrix, the file 802 may be represented with the following pointer pairs 816, which each point into the consensus sequence 808 and correspond to a part of the file 802:
P₁—(1:3)—this corresponds to ABA (see row 824 of the matrix 806);
P₂—(5:8)—this corresponds to ADCC (see row 824 of the matrix 806);
P₃—(0:7)—this corresponds to CABACADC (see row 826 of the matrix 806);
P₄—(0:2)—this corresponds to CAB (see row 828 of the matrix 806);and
P₅—(4:6)—this corresponds to CAD (see row 828 of the matrix 806).

The compressed file 818 includes P₁ . . . P₅ and the consensus sequence 808. This information allows the file to be decompressed into the file 802. More specifically, the file 802 is reconstructed by replacing each pointer in the list of pointers with the subsequence (letters or bits corresponding to the letters) of the consensus sequence 808 to which the pointers point. This process does not require the gaps to be considered as the pointer pairs 816 do not reference gaps but only reference the consensus sequence 808.

In one example and if desired, the compressed file 818 may be compressed with another compressor 820 (e.g., Hoffman Coding) to generate a compressed file 822, which has been compressed twice in this example. This allows the consensus sequence 808, which may be long, and/or the pointer pairs 816 to be compressed by the compressor 820 for additional space savings.

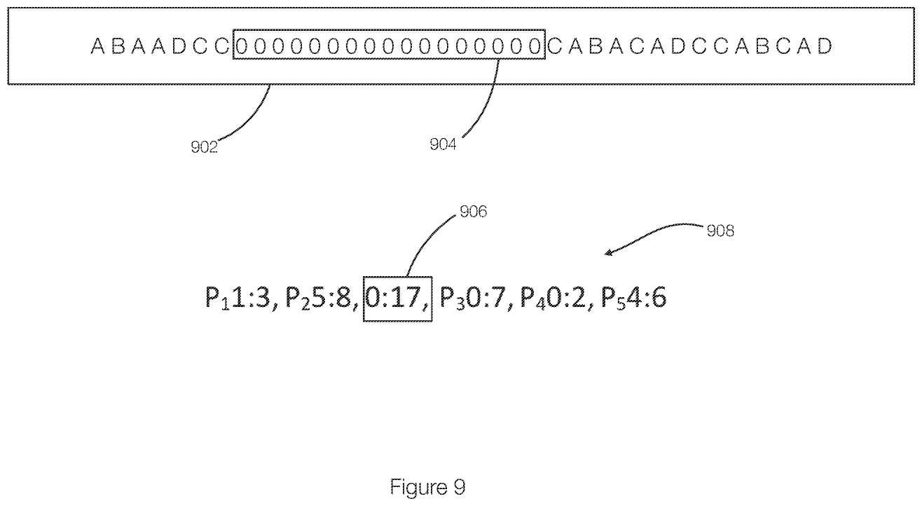

In one example, long 0 sequences (sequences of 0 bits) are not represented with a letter. Rather, long 0 sequences may be represented as a 0 sequence and a length. FIG. 9 discloses aspects of handling long 0 sequences in a file. FIG. 9 illustrates a file 902, which is similar to the file 802 but includes a zero sequence 904. In this example, sequencing the file 902 may result in the same matrix 206 as the zero sequence 904 may be omitted or handled differently. Thus, the pointer pairs for the file 904 are the same as discussed in FIG. 2.

In this example, the zero sequence 904 and its length are identified as a pair 906 and inserted into the pointer pairs 908 at the appropriate place (after P₂ and before P₃). The pair 906 represents a zero-sequence having a length of 17 bits—(0:17). One sequences (a sequence of 1 s) could be handled in a similar manner if present.

In one example, the actual data from the consensus sequence may be used in the pointer pairs instead of a pointer pair. More specifically, if the letters in the consensus sequence represent a small number of bits, it may conserve storage space to simply include the subsequence as present in the consensus matrix because the subsequence may take less space than the pointers (each pointer includes multiple bits).

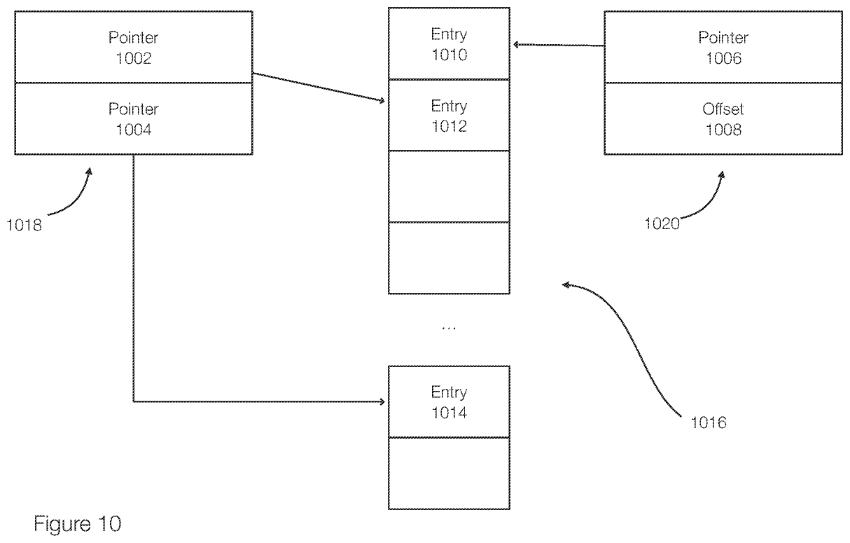

FIG. 10 discloses additional aspects of compressing data. FIG. 10 illustrates a consensus sequence 1016, which includes various entries that may be represented as a vector. FIG. 10 illustrates a pointer pair 1016, which includes pointer 1002 and pointer 1004. The pointer for points to entry 1012 and the pointer 1004 points to entry 1014. The pointer pair 1018 thus represents a portion of a file that has been compressed using a consensus sequence 1016. The pointer pair 1018 identifies a subsequence of the consensus sequence 1016.

FIG. 10 also illustrates a pointer pair 1006 that includes a pointer 1006 and an offset 1008. Using a pointer pair 1006 may be useful and may conserve space by eliminating the need to store a second pointer (the offset may consume less space than a pointer). Thus, the pointer pair 1020 identifies a starting entry 1010 and an offset 1008, which is the length of the subsequence identified by the pointer pair. Thus, the offset 1008 may require less space than the pointer 1004.

The length represented by the offset 1008 may also be represented using a variable length quantity (VLQ) to conserve or manage storage requirements. For example, the length of the sequence represented by the offset 1008 is less than 127, a single byte may be used. The most significant bit is used to identify whether other bytes are used to represent the length of the sequence. If the length is longer than 127, two bytes may be used as the offset.

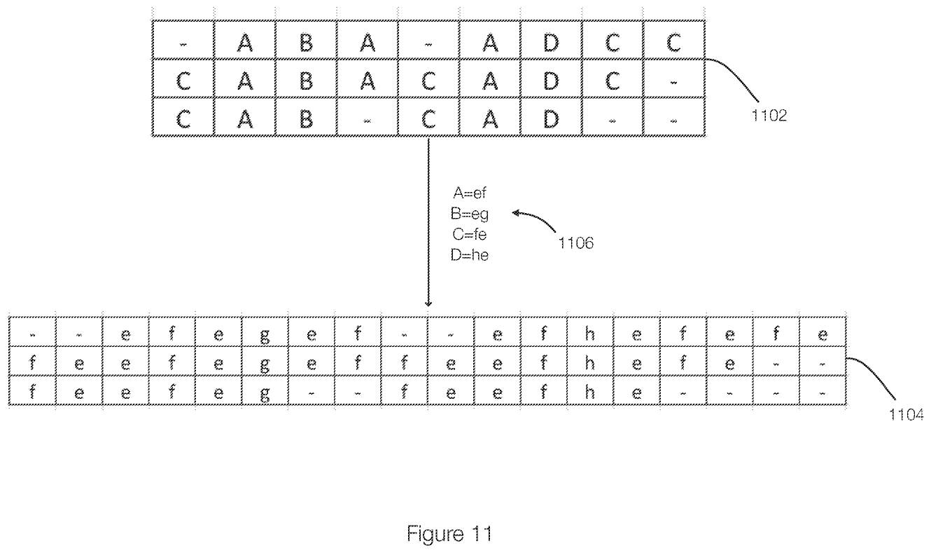

FIG. 11 discloses aspects of warm starting a compression operation. The warm start, for example, may be used in alignments performed by the alignment operation (e.g., a greedy splitting operation). FIG. 11 illustrates a matrix 1102, which is the same as the matrix 906. When the letter size is large (e.g., each letter represents 128 bits), the computation time to generate the matrix 1102 and compress the input file may be faster compared to when the letter size is smaller. In this example, the matrix 1102 resulted from processing a file.

Next, the letter sizes may be halved, thus the new letters 1106 are generated as A=ef, B=eg, C=fe, and D=he. This may result in the matrix 1104. More specifically, the matrix 1102 (or the associated alignment) may be generated as larger letter sizes is associated with quicker computation times. Thus, the matrix 1102 may be used as a prior for a subsequent alignment operation with iteratively reduced (e.g., halved) letter sizes. Thus, the matrix 1102 (or alignment information generated by the alignment engine) is used as a starting point for generating the matrix 1104.

Embodiments of the invention may also perform processing prior to aligning the file. For example, a size of a file may be large (e.g., terabytes). Compressing such a file may require significant amounts of RAM. As the letter size decreases or due to the size of the file or for other reasons, the available RAM may be insufficient. Embodiments of the invention may compress the file using hierarchical alignments.

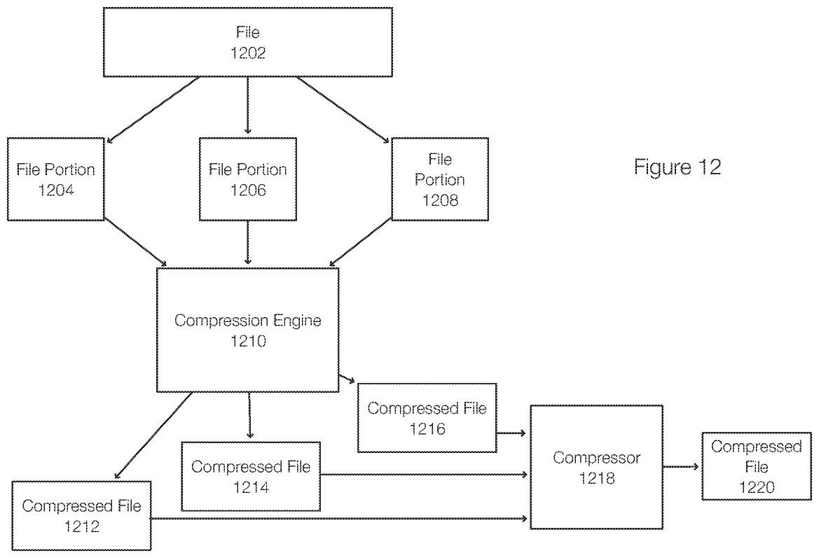

FIG. 12 discloses aspects of hierarchical alignment. FIG. 12 illustrates a file 1202 that may be large (e.g., terabytes). To accommodate existing resources, the file 1202 may divided into one or more portions, illustrated as portions 1204, 1206, 1208. These portions are then compressed (sequentially, concurrently, or using different compression engines) by the compression engine 1210 to generate, respectively, compressed files 1212, 1214, and 1216. A compressor 1218, which may be difference from the compression engine 1210 and use other compressors, may be used to compress the compressed files 1212, 1214, and 1216 into a single compressed file 1220. Thus, compresses files including a consensus sequence and pointer pairs are generated for each of the portions 1204, 1206, and 1208 and these compressed files are either compressed or concatenated into the compressed file 1220.

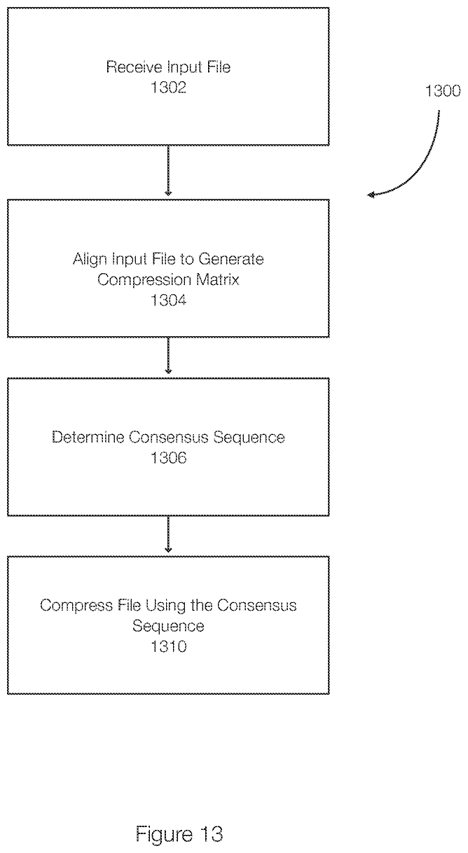

FIG. 13 discloses aspects of a method for compressing data. Initially, an input file is received 1302 in the method 1300 at a compression engine. The input file is then aligned 1304 or sequenced by an alignment engine to generate a compression matrix. Aligning a file may include splitting the file one or more times and/or adding gaps as necessary in order to generate a plurality of sequences that are aligned. The sequences are aligned, for example, when the sequences generated by splitting the file can be arranged in a matrix such that each column includes a single letter and/or gaps.

Next, the consensus sequence is determined 1306. This may be performed by flattening the matrix or by selecting, for each entry in the consensus sequence, the letter in the corresponding column of the compression matrix.

Once the consensus sequence is determined, the file is compressed 1308. This may include generating pointer pairs that point to the consensus sequence. Each pointer corresponds to a portion of the file. The file can be decompressed or reconstructed by concatenating the portions of the consensus sequence (or bits corresponding to the letters in the consensus sequence) correspond to the pointer pairs.

The computational requirements to compress files as discussed herein can be reduced, at the cost of compression efficiency. Examples of hyperparameters or parameters to consider include letter size (number of bits), number of initial splits of a file, minimal consensus length reduction required by the alignment engine (e.g., splitting operation), the size of initial chunks, and the number of stages where consensus sequences are aligned to a larger consensus sequence. Additionally, the role of the file (e.g., data base Kubernetes cluster) and block size used by the operating systems.

In one example, a glucose enantiomer (e.g., see FIG. 1A) can represent double representations. This allows a monosaccharide to include 16 representations (each OH can be viewed as a bit). Given these various representations, this allows an alphabet to be generated where each letter in the alphabet represents a particular configuration of the monosaccharide. In another example, it may be possible to set letter sizes based on a monosaccharide and/or on multiple monosaccharides. As a result, each monosaccharide in a polysaccharide can represent a sequence or other data.

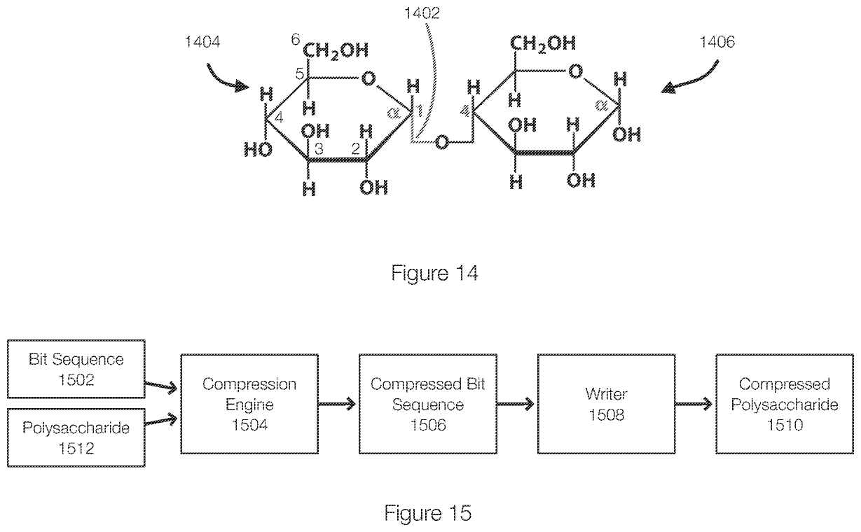

FIG. 14 further illustrates aspects of encoding data in a polysaccharide. FIG. 14 illustrates a bond 1402 between a glucose enantiomer (e.g., a monosaccharide) 1404 and a monosaccharide 1406. In one example, the glucose enantiomer 1404 includes carbons. The positions are labeled 1, 2, 3, 4, 5, 6, for explanation and ease of reference. A glycosidic bond can be formed at any of these bonds that includes an OH bond. Thus, the carbon at position 5 in FIG. 14 may be excluded from forming a glycosidic bond. In this example, the bond 1402 occurs between C1 of the monosaccharide 1404 and C4 of the monosaccharide 1406. As bonds are performed using different carbons (e.g., C1-C4, C2-C3), the geometry of the molecule may change. When storing data in polysaccharide form, it may be necessary to include information identifying which carbon is used to create a bond to the subsequent glucose enantiomer. By way of example only, the location or carbon used for a bond can be identified based on its position relative to the HC₂OH. In this example, the carbons are defined such that the carbon at HC₂OH is the last. The numbering could be different. The location of the bond between the glucose enantiomer 1404 and the glucose enantiomer 1406 may be identified in another manner. The bond 1402 can also be used to encode information and may allow for additional letters or a different alphabet of letters.

FIG. 15 discloses aspects of compressing data such that the data can be stored in polysaccharide storage. FIG. 15 represents multiple distinct methods. These methods, however, may involve some of the same or similar elements. One method relates to the bit sequence 1502 and another method relates to the polysaccharide 1512. With regard to the methods illustrated in FIG. 15, the method beginning with the bit sequence 1502 illustrates how data is compressed and stored on a polysaccharide. In this example, the polysaccharide is used to store compressed data.

Initially, a bit sequence 1502 is identified or received. The bit sequence 1502 can be compressed as discussed herein to generate a compression matrix and a consensus sequence. The compression engine 1504 may then generate a compressed bit sequence.

A polysaccharide writer 1508 can write the compressed bit sequence 1506 as a compressed polysaccharide 1510. A string of bits can be similarly compressed and stored in DNA storage.

In another example, also illustrated in FIG. 15, the input to the compression engine 1504 may be a polysaccharide 1512. More specifically in one example wherein the polysaccharide 1512 or virtual representation thereof is the input and is being compressed. In this example, letters may be assigned to the polysaccharide or to the glucose enantiomers and/or bonds. In this case, there may be original data that was compressed to generate the polysaccharide 1512 or the polysaccharide 1512 may simple represent an uncompressed sequence. However, the MSA used to generate the polysaccharide 1512 is distinct from the MSA or letters used to compress the polysaccharide. In the latter case, the letters may refer to specific configurations of a glucose enantiomer and/or an associated bond.

If the polysaccharide 1512 is a chain without branches, the compression engine 1504 compresses the simple polysaccharide 1512 as discussed herein.

In one example, a letter size may be selected that corresponds to a monosaccharide (or a glucose enantiomer) or to a plurality of monosaccharides. The letter size may also account for the bond between the monosaccharides. The bond information may be separate and may be compressed separately. To compress the polysaccharide, the polysaccharide can be read, if necessary, or the sequence may be known from when the polysaccharide was originally created. Using the letter size, the polysaccharide can be split and aligned. Once a compression matrix and consensus sequence are generated, the polysaccharide can be compressed and stored as a polysaccharide. The compressed representation of the polysaccharide 1512 is thus written new polysaccharide that corresponds to the compressed polysaccharide.

As discussed herein, the compressed data, such as the compressed polysaccharide 1510, may be associated with pointers, a pointer list, or the like. In one example, the pointers may be added at some end of the polysaccharide, stored as a different molecule, or the like.

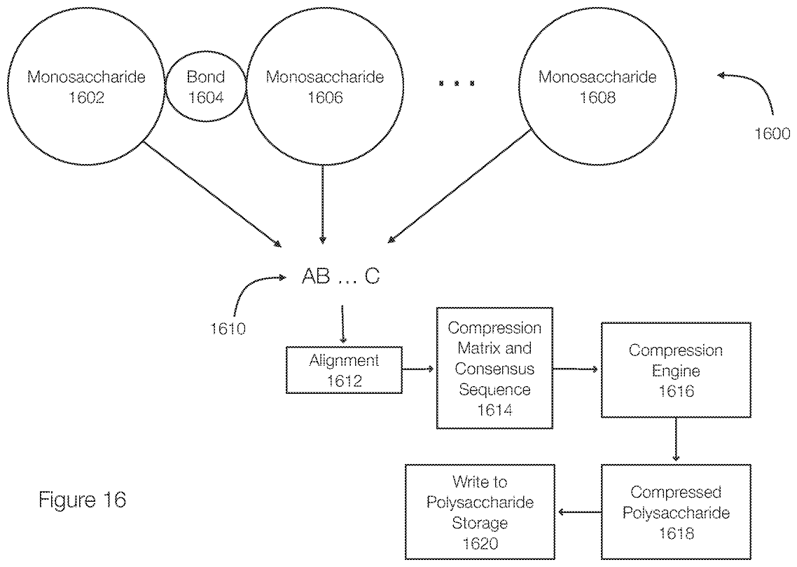

FIG. 16 discloses aspects of compressing a polysaccharide. FIG. 16 illustrates a polysaccharide 1600, which include a chain (a simple sequence with no branches) of glucose enantiomers (represented by monosaccharides 1602, 1606 and 1608). The bonds between the monosaccharides are represented by the bond 1604.

The monosaccharides can each be represented by a letter and a string of letters 1610 may be determined or identified, which corresponds to the polysaccharide 1600. Thus, the letter A corresponds to the monosaccharide 1602, the letter B corresponds to the monosaccharide 1606, and the letter C corresponds to the monosaccharide 1608 in this example. In another example, each of the monosaccharides could be represented by multiple letters (e.g., a smaller letter size). The bond 1604 may also be represented in a letter. Thus, the monosaccharide 1602 and the bond 1604 may be represented by the letter A. Because of the number of possible bonds, this may increase the alphabet used to represent the polysaccharide 1600. Alternatively, the bond 1604 may be represented and/or stored separately. Further, the bond may or may not be used to encode information. When used to encode data, the bond may be represented in the sequence 1610.

For example, assume that the monosaccharide represents 4 bits and that the original data letters are 8 bits each. Thus, 2 monosaccharides are required for each original data letter (excluding the bond). This represents a different alphabet to work on. Because the split in this example is on exact bit boundaries, when the bonds are considered, there is very different alignment between the original alphabet and the polysaccharide alphabet. When bonds are included, the MSA generates a different consensus matrix and a different consensus sequence. The result can be stored on a polysaccharide, which is different as it is compressed.

Once the sequence 1610 is generated, the sequence 1610 is aligned via the alignment 1612 to generate a compression matrix and a consensus sequence 1614. the sequence 1610 is then compressed by the compression engine 1616 to generate a compressed polysaccharide 1618. The compressed polysaccharide 1618 can be written 1620 to polysaccharide storage as a polysaccharide. In this example, the compressed polysaccharide 1618 is smaller than the polysaccharide 1600. The compressed polysaccharide 1618 may include pointers that point into the consensus sequence such that the original polysaccharide (or data) may be reconstructed as discussed herein. As previously stated, the pointers may be added to an end of one of the polysaccharide sequences, stored in a separate polysaccharide (or other form), or the like.

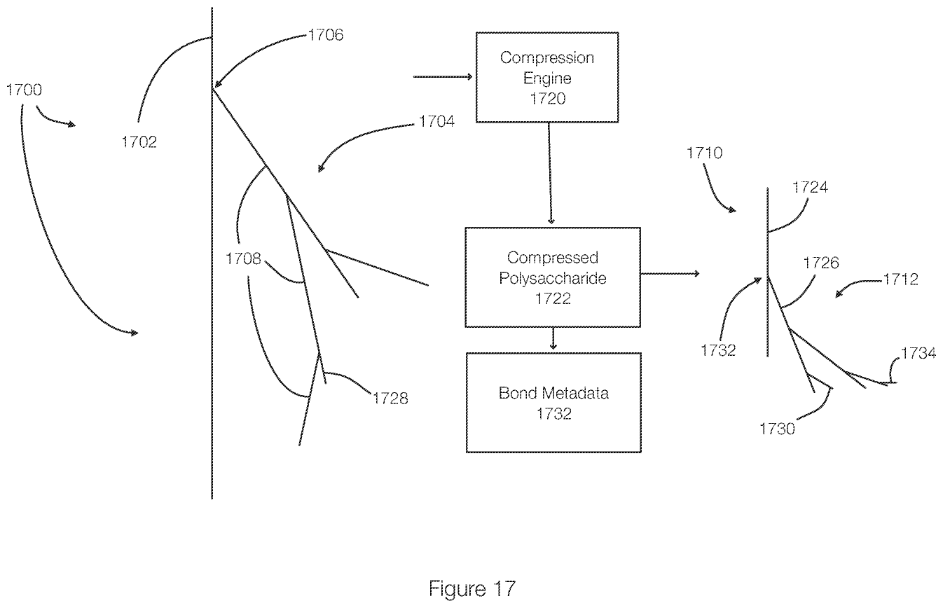

FIG. 17 discloses aspects of compressing a branched polysaccharide. FIG. 17 illustrates a branched polysaccharide 1700, which is a more simplified view of the polysaccharide 500 shown in FIG. 5. In one example, the polysaccharide 1700 (or data represented by or stored as the polysaccharide 1700 is compressed by the compression engine 1720 into a compressed polysaccharide 1722. The compressed polysaccharide 1722 may be written as the polysaccharide 1710. As illustrated, the polysaccharide 1710 is smaller in size than the polysaccharide 1700.

The polysaccharide 1700 includes a sequence 1702 and branches 1704. When reading the polysaccharide 1700, the polysaccharide 1700 may be read in a depth first manner. Thus, the first chain read is the sequence 1702. The sequence 1702 may be compressed as discussed herein. The compressed sequence 1724 represents the compressed chain 1702.

Next, the chain 1708 is identified and compressed. This is represented by the compressed sequence 1726. The sequence 1728 is compressed as the sequence 1730.

In one example, the bonds in the polysaccharide 1700, such as the bond 1706, represent a topology of the polysaccharide 1700. The bond 1706 may be, for example C1 to C4 of the relevant monosaccharides between the sequence 1702 and the sequence 1708. During compression, this bond may not be available in the polysaccharide 1710. In other words, the bond 1732, which corresponds to the bond 1706, may be different. The bond metadata 1732, which represents bonds or the topology of the polysaccharide 1700, may be incorporated into the polysaccharide 1710. This may be encoded as an additional branch that is added to the polysaccharide 1710 (e.g., the branch 1734). The branch 1734 may be the last branch read when reading the polysaccharide 1710 in a depth first manner. The topology could be written as a separate polysaccharide.

Returning to FIG. 15, the polysaccharide 1512 may be a branched polysaccharide. When reading the branched polysaccharide in a DFS (Depth First Search) manner, the compression matrix can be generated. Each run of the DFS search can be performed, in one example, without backtracking. Even though the polysaccharide can be read and reconstructed, embodiments of the invention include treating the polysaccharide as the data to be compressed. The alphabet used to perform MSA on the monosaccharides and/or their topology (bonds) is distinct from the alphabet used to generate the polysaccharide in the first place. Once the matrix is generated, the split subsequences are processed in order. This allows the consensus matrix, consensus sequence, and pointers to be generated.

In one example, when creating a consensus matrix (e.g., in the context of a branched polysaccharide), the matrix may include some sequences and may not include other sequences. For example, when processing a branch, the branch may present a sequence that is not present in the current consensus matrix. This provides at least two options. The new sequence can be added to the existing consensus matrix. This allows, once the matrix is complete, pointers to be used as discussed herein. When adding a new sequence, there is a possibility that part of the new sequence exists in the current matrix. For example, ABD is present in the matrix and a sequence ABCD is identified. This allows the existing matrix to be modified to accommodate the new sequence. Alternatively, columns could be added to the matrix. Each option has different implications on pointer management and resulting size. For example, this may impact how the branch is attached (where, how) to the polysaccharide

Once the consensus is finalized, the pointers are generated and stored in any chosen form, including as a polysaccharide if desired.

In bioinformatics, multiple sequence alignment (MSA) refers to a process or the result of sequence alignment of several biological sequences such as sugars, protein, DNA (deoxyribonucleic Acid), or RNA (ribonucleic acid). The input set of query sequences are assumed to have an evolutionary relationship. While the goal of MSA is to align the sequences, the output, in contrast to embodiments of the invention, may include multiple types of interactions including gaps, matches, and mismatches.

Appendix A includes an example of a Bacterial Foraging Optimization-Genetic Algorithm (BFO-GA) pseudocode and an example of an output of the BFO-GA algorithm. The BFO-GA, however, is not configured for compressing data, while embodiments of the invention relate to compressing data. Unlike BFO-GA and other MSA algorithms, the compression engine does not allow for mismatches and provides a positive score for subsequence matches above a certain length. Subsequence matches above a certain length facilitate the use of pointers during compression instead of the sequence itself.

The following is a discussion of aspects of example operating environments for various embodiments of the invention. This discussion is not intended to limit the scope of the invention, or the applicability of the embodiments, in any way.

In general, embodiments of the invention may be implemented in connection with systems, software, and components, that individually and/or collectively implement, and/or cause the implementation of, compression operations and/or data protection operations. Data protection operations which may include, but are not limited to, data replication operations, 10 replication operations, data read/write/delete operations, data deduplication operations, data backup operations, data restore operations, data cloning operations, data archiving operations, and disaster recovery operations. More generally, the scope of the invention embraces any operating environment in which the disclosed concepts may be useful.

At least some embodiments of the invention provide for the implementation of the disclosed functionality in existing backup platforms, examples of which include the Dell-EMC NetWorker and Avamar platforms and associated backup software, and storage environments such as the Dell-EMC DataDomain storage environment. In general, however, the scope of the invention is not limited to any particular data backup platform or data storage environment.

New and/or modified data collected and/or generated in connection with some embodiments, may be stored in a data protection environment that may take the form of a public or private cloud storage environment, an on-premises storage environment, and hybrid storage environments that include public and private elements. Any of these example storage environments, may be partly, or completely, virtualized. The storage environment may comprise, or consist of, a datacenter which is operable to service read, write, delete, backup, restore, and/or cloning, operations initiated by one or more clients or other elements of the operating environment. Where a backup comprises groups of data with different respective characteristics, that data may be allocated, and stored, to different respective targets in the storage environment, where the targets each correspond to a data group having one or more particular characteristics.

Example cloud computing environments, which may or may not be public, include storage environments that may provide data protection functionality for one or more clients. Another example of a cloud computing environment is one in which processing, data protection, and other, services may be performed on behalf of one or more clients. Some example cloud computing environments in connection with which embodiments of the invention may be employed include, but are not limited to, Microsoft Azure, Amazon AWS, Dell EMC Cloud Storage Services, and Google Cloud. More generally however, the scope of the invention is not limited to employment of any particular type or implementation of cloud computing environment.

In addition to the cloud environment, the operating environment may also include one or more clients that are capable of collecting, modifying, and creating, data. As such, a particular client may employ, or otherwise be associated with, one or more instances of each of one or more applications that perform such operations with respect to data. Such clients may comprise physical machines, virtual machines (VM), or containers.

Particularly, devices in the operating environment may take the form of software, physical machines, VMs, containers, or any combination of these, though no particular device implementation or configuration is required for any embodiment. Similarly, data protection system components such as databases, storage servers, storage volumes (LUNs), storage disks, replication services, backup servers, restore servers, backup clients, and restore clients, for example, may likewise take the form of software, physical machines, virtual machines (VM), or containers, though no particular component implementation is required for any embodiment.

As used herein, the terms ‘data’ and ‘file’ are intended to be broad in scope. Thus, these terms embrace, by way of example and not limitation, data segments such as may be produced by data stream segmentation processes, data chunks, data blocks, atomic data, emails, objects of any type, files of any type including media files, word processing files, spreadsheet files, and database files, as well as contacts, directories, sub-directories, volumes, images, logs, databases, multi-dimensional data, and any group of one or more of the foregoing.

It is noted that any of the disclosed processes, operations, methods, and/or any portion of any of these, may be performed in response to, as a result of, and/or, based upon, the performance of any preceding process(es), methods, and/or, operations. Correspondingly, performance of one or more processes, for example, may be a predicate or trigger to subsequent performance of one or more additional processes, operations, and/or methods. Thus, for example, the various processes that may make up a method may be linked together or otherwise associated with each other by way of relations such as the examples just noted. Finally, and while it is not required, the individual processes that make up the various example methods disclosed herein are, in some embodiments, performed in the specific sequence recited in those examples. In other embodiments, the individual processes that make up a disclosed method may be performed in a sequence other than the specific sequence recited.

Following are some further example embodiments of the invention. These are presented only by way of example and are not intended to limit the scope of the invention in any way. \

Embodiment 1. A method, comprising: receiving a polysaccharide, associating an alphabet with glucose enantiomers and/or bonds included in the polysaccharide, compressing the polysaccharide by recursively splitting and aligning letters in the alphabet generate a compression matrix, wherein the compression matrix represents polysaccharide, determining a consensus sequence from the compression matrix, and generating a compressed polysaccharide from the consensus sequence.

Embodiment 2. The method of embodiment 1, wherein the polysaccharide is a virtual polysaccharide, further comprising the compressed polysaccharide as a new polysaccharide.

Embodiment 3. The method of embodiment 1 and/or 2, further comprising generating pointers into the consensus sequence for the sequences in the compression matrix.

Embodiment 4. A method, comprising: reading a polysaccharide or a virtual manifestation of the polysaccharide to generate at least one sequence, compressing the sequence of glucose enantiomers by recursively splitting and aligning the sequence using letters associated with the polysaccharide to generate a compression matrix, wherein each letter represents at least a glucose enantiomer and/or a bond, determining a consensus sequence from the compression matrix, and generating a compressed polysaccharide from the consensus sequence.

Embodiment 5. The method of embodiment 4, wherein the polysaccharide comprises a simple sequence.

Embodiment 6. The method of embodiment 4 and/or 5, wherein the polysaccharide comprises a branched sequence.

Embodiment 7. The method of embodiment 4, 5, and/or 6, further comprising reading the branched sequence in a depth first manner.

Embodiment 8. The method of embodiment 4, 5, 6, and/or 7, further comprising compressing a first sequence obtained from reading the branched sequenced in the depth first manner.

Embodiment 9. The method of embodiment 4, 5, 6, 7, and/or 8, further comprising compressing a first branch of the first sequence.

Embodiment 10. The method of embodiment 4, 5, 6, 7, 8, and/or 9, further comprising determining a topology of the branched polysaccharide.

Embodiment 11. The method of embodiment 4, 5, 6, 7, 8, 9, and/or 10, further comprising storing the topology with the compressed polysaccharide, wherein the topology identifies locations of branches and bonds between monosaccharides at the branches.

Embodiment 12. The method of embodiment 4, 5, 6, 7, 8, 9, 10, and/or 11, wherein the topology encodes data.

Embodiment 13. The method of embodiment 4, 5, 6, 7, 8, 9, 10, 11, and/or 12, further comprising storing the compressed polysaccharide in polysaccharide form.

Embodiment 14. A method for performing any of the operations, methods, or processes, or any portion of any of these, disclosed herein.

Embodiment 15. A non-transitory storage medium having stored therein instructions that are executable by one or more hardware processors to perform operations comprising the operations of any one or more of embodiments 1-14.

The embodiments disclosed herein may include the use of a special purpose or general-purpose computer including various computer hardware or software modules, as discussed in greater detail below. A computer may include a processor and computer storage media carrying instructions that, when executed by the processor and/or caused to be executed by the processor, perform any one or more of the methods disclosed herein, or any part(s) of any method disclosed.

As indicated above, embodiments within the scope of the present invention also include computer storage media, which are physical media for carrying or having computer-executable instructions or data structures stored thereon. Such computer storage media may be any available physical media that may be accessed by a general purpose or special purpose computer.

By way of example, and not limitation, such computer storage media may comprise hardware storage such as solid state disk/device (SSD), RAM, ROM, EEPROM, CD-ROM, flash memory, phase-change memory (“PCM”), or other optical disk storage, magnetic disk storage or other magnetic storage devices, or any other hardware storage devices which may be used to store program code in the form of computer-executable instructions or data structures, which may be accessed and executed by a general-purpose or special-purpose computer system to implement the disclosed functionality of the invention. Combinations of the above should also be included within the scope of computer storage media. Such media are also examples of non-transitory storage media, and non-transitory storage media also embraces cloud-based storage systems and structures, although the scope of the invention is not limited to these examples of non-transitory storage media.

Computer-executable instructions comprise, for example, instructions and data which, when executed, cause a general purpose computer, special purpose computer, or special purpose processing device to perform a certain function or group of functions. As such, some embodiments of the invention may be downloadable to one or more systems or devices, for example, from a website, mesh topology, or other source. As well, the scope of the invention embraces any hardware system or device that comprises an instance of an application that comprises the disclosed executable instructions.

Although the subject matter has been described in language specific to structural features and/or methodological acts, it is to be understood that the subject matter defined in the appended claims is not necessarily limited to the specific features or acts described above. Rather, the specific features and acts disclosed herein are disclosed as example forms of implementing the claims.

As used herein, the term ‘module’ or ‘component’ may refer to software objects or routines that execute on the computing system. The different components, modules, engines, and services described herein may be implemented as objects or processes that execute on the computing system, for example, as separate threads. While the system and methods described herein may be implemented in software, implementations in hardware or a combination of software and hardware are also possible and contemplated. In the present disclosure, a ‘computing entity’ may be any computing system as previously defined herein, or any module or combination of modules running on a computing system.

In at least some instances, a hardware processor is provided that is operable to carry out executable instructions for performing a method or process, such as the methods and processes disclosed herein. The hardware processor may or may not comprise an element of other hardware, such as the computing devices and systems disclosed herein.

In terms of computing environments, embodiments of the invention may be performed in client-server environments, whether network or local environments, or in any other suitable environment. Suitable operating environments for at least some embodiments of the invention include cloud computing environments where one or more of a client, server, or other machine may reside and operate in a cloud environment.

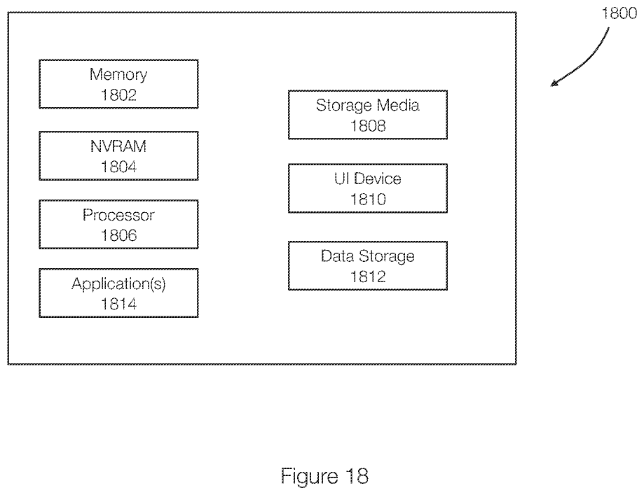

With reference briefly now to FIG. 18, any one or more of the entities disclosed, or implied, by the Figures and/or elsewhere herein, may take the form of, or include, or be implemented on, or hosted by, a physical computing device, one example of which is denoted at 1800. As well, where any of the aforementioned elements comprise or consist of a virtual machine (VM), that VM may constitute a virtualization of any combination of the physical components disclosed in FIG. 18.

In the example of FIG. 18, the physical computing device 1800 includes a memory 1802 which may include one, some, or all, of random access memory (RAM), non-volatile memory (NVM) 1804 such as NVRAM for example, read-only memory (ROM), and persistent memory, one or more hardware processors 1806, non-transitory storage media 1808, UI device 1810, and data storage 1812, one example of which is polysaccharide storage media. One or more of the memory components 1802 of the physical computing device 1800 may take the form of solid state device (SSD) storage. As well, one or more applications 1814 may be provided that comprise instructions executable by one or more hardware processors 1806 to perform any of the operations, or portions thereof, disclosed herein.

Such executable instructions may take various forms including, for example, instructions executable to perform any method or portion thereof disclosed herein, and/or executable by/at any of a storage site, whether on-premises at an enterprise, or a cloud computing site, client, datacenter, data protection site including a cloud storage site, or backup server, to perform any of the functions disclosed herein. As well, such instructions may be executable to perform any of the other operations and methods, and any portions thereof, disclosed herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
receiving a polysaccharide;
associating an alphabet with glucose enantiomers and/or bonds included in the polysaccharide;
compressing the polysaccharide by recursively splitting and aligning letters in the alphabet generate a compression matrix, wherein the compression matrix represents polysaccharide;
determining a consensus sequence from the compression matrix; and
generating a compressed polysaccharide from the consensus sequence.

2. The method of claim 1, wherein the polysaccharide is a virtual polysaccharide, further comprising the compressed polysaccharide as a new polysaccharide.

3. The method of claim 1, further comprising generating pointers into the consensus sequence for the sequences in the compression matrix.

4. A method comprising:
reading a polysaccharide or a virtual manifestation of the polysaccharide to generate at least one sequence;
compressing the sequence of glucose enantiomers by recursively splitting and aligning the sequence using letters associated with the polysaccharide to generate a compression matrix, wherein each letter represents at least a glucose enantiomer and/or a bond;
determining a consensus sequence from the compression matrix; and
generating a compressed polysaccharide from the consensus sequence.

5. The method of claim 4, wherein the polysaccharide comprises a simple sequence.

6. The method of claim 4, wherein the polysaccharide comprises a branched sequence.

7. The method of claim 6, further comprising reading the branched sequence in a depth first manner.

8. The method of claim 7, further comprising compressing a first sequence obtained from reading the branched sequenced in the depth first manner.

9. The method of claim 8, further comprising compressing a first branch of the first sequence.

10. The method of claim 9, further comprising determining a topology of the branched polysaccharide.

11. The method of claim 10, further comprising storing the topology with the compressed polysaccharide, wherein the topology identifies locations of branches and bonds between monosaccharides at the branches.

12. The method of claim 9, wherein the topology encodes data.

13. The method of claim 4, further comprising storing the compressed polysaccharide in polysaccharide form.

14. A non-transitory storage medium having stored therein instructions that are executable by one or more hardware processors to perform operations comprising:
reading a polysaccharide or a virtual manifestation of the polysaccharide to generate at least one sequence;
compressing the sequence of glucose enantiomers by recursively splitting and aligning the sequence using letters associated with the polysaccharide to generate a compression matrix, wherein each letter represents at least a glucose enantiomer and/or a bond;
determining a consensus sequence from the compression matrix; and
generating a compressed polysaccharide from the consensus sequence.

15. The non-transitory storage medium of claim 14, wherein the polysaccharide comprises a simple sequence.

16. The non-transitory storage medium of claim 14, wherein the polysaccharide comprises a branched sequence.

17. The non-transitory storage medium of claim 16, further comprising reading the branched sequence in a depth first manner.

18. The non-transitory storage medium of claim 17, further comprising compressing a first sequence obtained from reading the branched sequenced in the depth first manner.

19. The non-transitory storage medium of claim 8, further comprising compressing a first branch of the first sequence and determining a topology of the branched polysaccharide.

20. The method of claim 10, further comprising storing the topology with the compressed polysaccharide, wherein the topology identifies locations of branches and bonds between monosaccharides at the branches.
